(19) United States
(12) Patent Application Publication
(10) Pub. No.: US 2024/0368644 A1
(43) Pub. Date: Nov. 7, 2024

(54) METHODS TO PRODUCE THERAPEUTIC FORMULATIONS COMPRISING HYDROXYBUTIRATE AND HYDROXYVALERATE, THERAPEUTIC FORMULATIONS AND USES THEREOF

(71) Applicants: PHB Industrial S.A., Serrana, San Paulo (BR); Vitanav, Inc., Washington, DC (US)

(72) Inventors: Roberto Vianna NONATO, Serrana, Sao Paulo (BR); Devon PRICE, Fulton, MD (US); Peter BAYNE, Washington, DC (US)

(73) Assignees: PHB Industrial S.A., Serrana, San Paulo (BR); Vitanav, Inc., Washington, DC (US)

(21) Appl. No.: 18/574,355

(22) PCT Filed: Jun. 23, 2022
(86) PCT No.: PCT/US2022/034675

§ 371 Date: Dec. 27, 2023

Related U.S. Application Data
(60) Provisional application No. 63/215,711, filed on Jun. 28, 2021.

(51) Int. Cl. C12P 7/62; A61K 31/047; A61K 31/19; A61P 3/00; A61P 3/08

(52) U.S. Cl. CPC C12P 7/62; A61K 31/047; A61K 31/19; A61P 3/00; A61P 3/08

(57) ABSTRACT

A process for producing optically active (R)-3-hydroxybutyrate and (R)-3-hydroxyvalerate mixtures, where the ratio between them is defined by the composition of PHB-co-HV, used as raw material for the production process, formulations containing the mixtures, and uses of the formulations. A use or method of treating a subject having a metabolic disorder, comprising administering optically active mixtures of (R)-3-hydroxybutyrate and (R)-3-hydroxyvalerate, their respective salts or esters derived therefrom, obtained according to the process of the present disclosure at a therapeutically effective amount to treat at least one symptom of the metabolic disorder.

### CROSS-REFERENCE TO RELATED APPLICATION

This application claims the benefit of U.S. Provisional Application No. 63/215,711 filed on Jun. 28, 2021, the disclosures of which are incorporated herein in their entirety by reference.

### FIELD

The invention, the subject of this patent application, relates to a process for producing optically active (R)-3-hydroxybutyrate and (R)-3-hydroxyvalerate mixtures, where the ratio between them is defined by the composition of PHB-co-HV, used as raw material for the production process, formulations containing the mixtures, and uses of the formulations.

### BACKGROUND

The brain has a huge demand for energy. However, as it doesn't have any energy storage, it requires a continuous supply of substances that can generate this energy. In a normal situation, at rest and under a balanced diet, the brain consumes about 120 g of glucose per day, which corresponds to an energy input of about 420 kcal (1760 kJ), representing about 60% of the whole glucose usage by the body. It is estimated that 60% to 70% of this energy is used to energize the transport mechanisms that maintain the membrane potential necessary for the transmission of nerve impulses.

Glucose, however, is not just a source of energy. The brain also needs to synthesize neurotransmitters and their receptors to propagate nerve impulses. Glucose, in this respect, plays a key role for anaplerosis, that is, it provides intermediate substrates to metabolic pathways responsible for the production of various compounds that are important for cell functioning. In individuals with problems involving the glucose metabolism, caused by deficiencies in its transport into the cell or in some other specific pathway, these two roles, energy and anaplerosis, may be negatively affected.

The energy deficit, associated or not with the low availability of metabolic intermediates, is closely associated, as a cause or effect, with the degeneration of the central nervous system. In several diseases, such as Alzheimer's, epilepsy, Huntington's, Parkinson's and physical trauma, the benefit of so-called ketogenic diets has been demonstrated. This type of diet is based on a drastic restriction on carbohydrate consumption. Typically, a ketogenic diet has a weight ratio of 3 to 4 pats of oils and fats to 1 part of the sum of carbohydrates and proteins. Its effect is to force a metabolic situation where these oils and fats are broken down, leading to the production of ketone bodies, which are capable of providing an alternative source of energy to different tissues, including nerve cells. In this way, they replace glucose in its first metabolic role.

However, the ketone bodies produced as a result of the ketogenic diet, 3-hydroxybutyrate and acetoacetate, have an even carbon chain (C4-KB). This is due to the near absence of natural edible oils with odd-numbered carbon chains. Even-chain ketone bodies provide energy to the brain, but they are not anaplerotic, that is, they are not able to restore adequate levels of various compounds important to nerve cells, such as neurotransmitters. Odd-chain ketone bodies, 3-hydroxyvalerate and 3-ketovalerate (C5-KB), which would have this property, are virtually absent in the bloodstream of individuals on the most common ketogenic diets. Thus, the replacement of metabolic intermediates still needs glucose, which, in a ketogenic situation, as in ketogenic diets, is mainly supplied by the liver through a specific metabolic pathway, neoglycogenesis. However, if the cellular assimilation of glucose is impaired due to any of the various pathologies that affect it, this glucose produced by the liver will have little or no effect. On the contrary, it can cause problems and significantly diminish the potential benefits of the ketogenic diet.

Trying to get around this problem, several studies have been demonstrating the potential of using triheptanoin in ketogenic diets. Triheptanoin is an artificial triglyceride formed by three heptanoate chains linked to a glycerol molecule. As heptanoate has an odd carbon chain, it has anaplerotic properties—that is, it can replenish the pool of intermediate metabolites in the TCA cycle. Unlike even-chain fatty acids, metabolized only to acetyl-CoA, triheptanoin can both provide acetyl-CoA, aimed at energy production and propionyl-CoA, which can also serve in the construction of intermediates.

In therapeutic applications, triheptanoin was initially used in patients with disorders in the oxidation of long-chain fatty acids. The first demonstration of the possible benefit of triheptanoin for brain energy deficit came from a patient with pyruvate carboxylase deficiency, a serious metabolic disease that affects anaplerosis in the brain (Roe et al., 2002). In another study, triheptanoin has been shown to decrease paroxysmal non-epileptic manifestations by 90% in patients with glucose transporter 1 deficiency syndrome (GLUT1), a disease that affects glucose transport in the brain. (Mochel et al., 2016). Magnetic resonance spectroscopy studies also indicated that triheptanoin was able to correct bioenergetics in the brain of patients with Huntington's disease. These and other studies indicate that triheptanoin, applied in ketogenic diets, may be a treatment for deficit in cerebral energy and altered anaplerosis (Mochel, 2017, Wehbe and Tucci, 2020).

The routes taken by triheptanoin to reach the brain are well explored (Marin-Valencia, 2013): conversion to heptanoate by the digestive system and conversion to C5 ketone bodies (C5-KB) by the liver. Both heptanoate and C5-KB are then absorbed by the nervous system, specifically by astrocytes, through simple diffusion (in the case of heptanoate) or by means of a specific active carrier (MCT1, in the case of C5-KB). Astrocytes then break down these two types of molecules, generating both acetyl-CoA and propionyl-CoA, used for energy generation and intermediates in the TCA cycle, which are then shared with neurons.

Ketogenic diets, however, have some disadvantages. They can lead to increased levels of cholesterol and triglycerides, cause gastric/intestinal dysfunction, and are still poorly palatable to many people. Fatty acids derived from oils inserted in the diet are absorbed by different cells only by diffusion, which is a slow process; thus, they depend on liver activity, which converts them into ketone bodies.

The direct supply of ketone bodies, in the form of salts, acids or esters, leads to a metabolic effect very similar to that of mild ketosis, without, however, presenting the problems derived from the ketogenic diet. Several products available on the market present themselves as capable of supplying ketone bodies directly to the body, without the need to follow strict diets. Most of them are formulas based on salts of 3-hydroxybutyrate, 3-hydroxybutyric acid or derived esters with 1,3-butanediol (these esters, which are more expensive than the salts, have shown much more significative results). Marketed as nutritional supplements, these products are aimed at better sports and cognitive performance.

Both 3-hydroxybutyrate and 1,3-butanediol are molecules that have a chiral center, that is, they exist in two structural conformations, R and S. As they present an effect on the rotation of polarized light, they are called optical isomers, or optically active isomers. As in many other cases (such as glucose itself), only one of the isomeric forms is preferred in the metabolic pathways. Due to the lower production cost, though, most commercial products are based on a mixture of the two isomers, called a racemic mixture, which leads to less availability and speed of metabolization of the active ingredients, as well as an unwanted accumulation of the S isomer, not consumed at the same speed.

Several routes are possible to obtain optically active 3-hydroxybutyrate. Purely chemical processes were proposed, as in Noyori et al., (J. Am. Chem. Soc., 1987, 109 (19), pp 5856-5858) and EP-0855935 (Van Brussel), however in both routes, it was necessary to use high value chiral catalysts and the resultant product was of relatively low optical purity. Fermentative processes, in which direct production of (R)-3-hydroxybutyrate is sought, has been described, as in Park, SJ (Appl Biochem Biotechnol 2004). This production route, however, still has restrictions for commercial application, due to several reasons: it makes use of recombinant strains, which can restrict its use as a nutritional supplement; the concentrations achieved are low, which makes the recovery/concentration/purification process of the final product more complicated and costly; the presence of cellular compounds from the producing microorganism, even in low concentrations in the final product, may have allergenic implications that hinder its application as a nutritional supplement.

Optical resolution of racemic mixtures, obtained by synthesizing 3-hydroxybutyrate through simple chemical routes, may represent an option. For this purpose, enzymes are used, such as described in U.S. Pat. No. 7,485,452 (Hwang). This route, however, faces the difficulty of obtaining specific enzymes of high efficiency or suffers from low specificity of commercial enzymes.

Several authors described processes for the synthesis of (R)-3-hydroxybutyrate, its salts and esters, based on the degradation of polyhydroxyalkanoates (PHAs), in particular polyhydroxybutyrate (PHB). Lee et al. describes a process for the production of R-3-hydroxybutyrate alkyl ester using PHB produced by fermentation and purified by digesting the biomass with NaOH and detergent, or by extraction with chloroform. The purified PHB is dissolved in dichloroethane and subjected to an esterification reaction with methanol, ethanol or propanol, using hydrochloric acid as a catalyst. After heating for several hours at the boiling temperature of the mixture, the medium is neutralized, alcohol is removed, and the final product is obtained by vacuum fractional distillation. A similar process is described by DeRoo et al. and U.S. Pat. No. 5,107,016.

Various publications have described solvent extraction of PHB from different sources, such as plant biomass or microorganisms, as for instance EP01853713 (Mantellato) and EP01687436 (Mantellato). The procedures adopted in these publications, however, aim to obtain PHB with high purity and very low residual solvent, which is necessary when it is intended to be used in the thermoplastics industry. This need for high purity PHB makes the extraction/purification process relatively complex, involving several steps and operations. Similar purity is necessary when using PHB as raw material in the synthesis of (R)-3-hydroxybutyrate, to avoid contaminating the product with the solvents used. Consequently, the PHB extraction/purification step becomes much more complex than the synthesis/purification of the final products.

Processes for the production of (R)-3-hydroxybutyrate starting directly from biomass containing PHB, without any previous purification, such as the one in the US patent 2003/0162851 (Zhong), in our experience have quite bad results, both in efficiency, for losing part of the reagents in secondary reactions, and for the purification of the final product, hampered by the large amount of by-products and cellular waste.

A slightly different route from the ones proposed above makes use of the very degrading enzymes of the microorganism used in the production of PHA to break the polymer chain into monomers, as described in the U.S. Pat. No. 6,472,188 (Lee). This process, however, generates several by-products and final product purification is difficult, with low yields.

A very interesting process for obtaining 3-hydroxybutyrate esters from PHB is described as example 1 in European patent EP 2 984 066. In this example, a reaction mixture is presented containing only an alcohol, PHB and sulfuric acid as a catalyst. After the reaction and subsequent neutralization of the medium, the ester which is produced is separated by distillation. The non-use of other solvents in the reaction, such as described in U.S. Pat. No. 5,107,016, represents an undeniable advantage, both in relation to the small number of purification steps required and the purity level of the product, especially when considering use as a nutritional supplement. In our experience, this procedure works very well with purified PHB, but not with crude biomass containing PHB, due to the formation of several by-products when using the latter as a raw material. The direct use of biomass containing PHB, in comparison to the use of purified PHB, could represent, however, an enormous economic advantage, considering the complexity of the extraction/purification processes. Therefore, it is necessary to develop a simpler PHB extraction/purification procedure.

The European patent EP 2 984 066, in our view, presents the best solution for the synthesis of (R)-3-hydroxybutyrate, however it demands the use of high purity PHB as raw material to be successful. An interesting economic evaluation of PHB purification methods for this purpose is presented by Choi et al. In this article, two methods of purifying PHB from biomass are compared, one by solvent extraction (chloroform), the other by digestion with NaOH, the result being frankly favorable to the digestion of biomass with NaOH. Here, we have two problems: either a solvent is introduced into the process, or a large volume of water is introduced, which is necessary for the digestion of biomass. An additional solvent represents an obstacle in terms of product quality (chloroform, for example, would have great difficulties in the approval for use in food supplements, even in very low concentrations). Water, on the other hand, is detrimental to the yield of the esterification reaction and needs to be removed, increasing the costs of the process.

Regarding the use of 3-hydroxybutyrate in ketogenic diet formulations, either as an optically active isomer or racemic mixtures, ketogenic nutritional supplements currently on the market make use solely of even chain carbon compounds (3-hydroxybutyrate or 1,3-butanediol). Consequently, such supplements have effect only on energy metabolism: they are great substitutes for glucose, but only in the aspect of supplying energy. None of these products have any anaplerotic effect. They are not able to supply the cellular demand for intermediate compounds in the TCA cycle. Thus, there is a long-felt and unmet need for a process, composition and method of producing and providing a mixture of compounds that can meet the cellular demand for energy and restoring adequate levels of important metabolic intermediates.

### SUMMARY OF THE INVENTION

The present disclosure includes a process for purification of PHB-co-HV prior to the esterification reaction carried out by a low-cost enzymatic process. This makes the entire PHB extraction/purification process much simpler and more economical, in addition to leading to a final product with better purity, with no risk of contamination with solvents or reagents unrelated to the synthesis process.

The present disclosure includes compositions containing the mixture (R)-3-hydroxybutyrate, (R)-3-hydroxyvalerate and (R)-1,3 butanediol that are suitable for human consumption and capable both of meeting the cellular demand for energy and restoring adequate levels of important metabolic intermediates.

The present disclosure includes methods and uses of these compositions as supplements for the treatment of metabolic disorders, particularly those involving brain energy deficit from reduced glucose absorption capacity and aneplerotic deficiency, such as insulin resistance, glucose transporter 1 deficiency, diabetes, and central nervous system disorders, like Huntington's disease, pyruvate carboxylase deficiency, Alzheimer's disease, Parkinson's disease, and epilepsy.

Other features and characteristics of the subject matter of this disclosure, as well as the methods of operation, functions of related elements of structure and the combination of parts, and economies of manufacture, will become more apparent upon consideration of the following description and the appended claims, all of which form a part of this specification.

### BRIEF DESCRIPTION OF THE DRAWINGS

Various features of examples and embodiments in accordance with the principles described herein may be more readily understood with reference to the following detailed description taken in conjunction with the accompanying drawings, where like reference numerals designate like structural elements, and in which:

FIG. 1 represents the results of administration of (D)-β-hydroxybutyric acid, (D)-β-hydroxyvaleric acid and (D)-1,3 butanediol over the course of a 60-minute period in various ratios in a subject at rest; and

FIG. 2 represents the expected ketosis for a subject at rest with a 0.13 g/kg dose of (D)-β-hydroxybutyric acid, (D)-β-hydroxyvaleric acid and (D)-1,3 butanediol over time for the periods shown.

FIG. 3 is an electroencephalogram (EEG) of a female human test subject aged 46 before administration of the mixture (top) and after administration of the mixture (bottom).

FIG. 4 shows Alpha Relative Power comparing baseline record and after consuming the mixture.

FIG. 5 shows Gamma Relative Power comparing baseline record and after consuming the mixture.

FIG. 6 shows Delta Relative Power comparing baseline record and after consuming the mixture.

### DETAILED DESCRIPTION OF THE INVENTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described. All references mentioned herein are incorporated herein by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

As used herein, the terms “about” and “approximately” are used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain compositions, elements, excipients, ingredients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date.

As used herein, the phrase “substantially no” may refer to a composition containing less than 1, 0.9, 0.8, 0.7, 0.6, 0.5, 0.4, 0.3, 0.2, 0.1, 0.09, 0.08, 0.07, 0.06, 0.05, 0.04, 0.03, 0.02, or 0.01 wt % of a specified ingredient. In some aspects, the phrase “substantially no” may refer to a composition containing trace amounts of a specified ingredient. In some aspects, the phrase “substantially no” may refer to a composition containing a specified ingredient below a level of detection.

In some aspects, the present disclosure includes using an enantiomerically pure form of a compound, e.g., greater than 95, 96, 97, 98, 99, or 99.5% enantiomerically pure.

Throughout this description, the preferred embodiments and examples provided herein should be considered as exemplary, rather than as limitations, of the present invention.

As used herein, the terms “administer” and “administration” will include self-administration, ingestion, or consumption by a subject. In other words, the terms will include methods that result in consumption of the disclosed products by a subject. As such, methods of the present disclosure will include making, using, selling, offering for sale, importing, or exporting any of the products or compositions of the present disclosure intended for consumption or use in producing a consumable product.

In one aspect, the present disclosure provides a process for the production of nutritional formulations involving both energetic and anaplerotic agents, as well as to a method for the treatment of neurodegenerative diseases related to disorders in the glucose metabolic pathways, involving the administration of mixtures of the energy supplier (R)-3-hydroxybutyrate and the anaplerotic agent (R)-3-hydroxyvalerate, their salts or esters.

An excellent alternative for the production of nutritional formulations involving both energetic and anaplerotic agents is the simultaneous production of optically active (R)-3-hydroxybutyrate and (R)-3-hydroxyvalerate from the controlled degradation of polyhydroxyalkanoates, or PHAs, formed with polymeric chain involving monomers with an even number of carbons, interspersed with monomers with an odd number of carbons.

PHAs are polyesters naturally synthesized by many living beings, with more than 170 different molecules described in the literature. The main commercial interest for PHAs involves applications in the plastics industry, as they are polyesters with thermoplastic, natural and biodegradable properties. The chemical structure of PHAs can be described as a linear polymeric chain, formed by repetitions of the following unit:

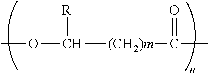

where R is an alkyl or alkenyl group of variable length and m and n are integer numbers. Regarding the above-mentioned polymers, R and m assume the following values:
PHB: R=CH₃, m=1
PHB-V: R=CH3 or CH3-CH2-, m=1
P4HB: R=H, m=2
P3HB-4HB: R=H or CH3, m=1 or 2
PHHx: R=CH₃—CH₂—CH₂—, m=1

Particularly relevant to the present invention is polyhydroxybutyrate-co-hydroxyvalerate, or PHB-co-HV, since it is a linear copolymer, formed by the repetition of the 3-hydroxybutyrate unit interspersed with 3-hydroxyvalerate units. Commercially available for several years, PHB-co-HV is produced by fermentation, using as raw materials sugars or vegetable oils and a precursor selected from odd carbon number substances, such as propionic or valeric acid. With a careful balance of nutrients and carbon sources, the polymers accumulate intracellularly by the producing microorganism and can be further harvested and purified.

Document BR 102018074086-5 describes a process for obtaining optically active 3-hydroxybutyrate alkyl esters. Through the teachings contained in this document, it is possible to obtain, for example, ethyl-(R)-3-hydroxybutyrate, using as raw material bacterial biomass containing polyhydroxybutyrate (PHB). Ethyl-(R)-3-hydroxybutyrate is a precursor that is easily converted to 3-(R)-hydroxybutyric acid by adding an alkali such as NaOH or KOH.

The document PI 9103116-8 describes a PHA production process, where the polymer composition can be controlled by the addition of precursors. Of special interest is the possibility of synthesizing homopolymer polyhydroxybutyrate, using exclusively cane sugar (PHB) and the polyhydroxybutyrate-co-hydroxyvalerate copolymer (PHB-co-HV), produced by mixing sugars and an odd carbon chain substance, such as propionic or valeric acids.

Applying the teachings of both documents, we developed an innovative process, through which it is possible to obtain a mixture of ethyl-(R)-3-hydroxybutyrate and ethyl-(R)-3-hydroxyvalerate with high purity, where the ratio between both is determined by the content of 3-hydroxyvalerate in the PHB-co-HV polymer. This content, in turn, is determined by the fermentative conditions under which the PHB-co-HV was produced.

In the invention described here, we developed a fermentation protocol using strains of the bacteria Azohydromonas lata DSM 1122 (formerly Alcaligenes latus DSM1122) or Cupriavidus necator DSM 545 (formerly Alcaligenes eutrophus DSM 545), cultivated under aerobic conditions, using sugarcane molasses as a source of main carbon and propionic acid or valeric acid as precursor of hydroxyvalerate units. The proportion between molasses and propionic or valeric acid almost directly defines the proportion of HV units in the final polymer PHB-co-HV.

A simple process of purification of PHB-co-HV, using a proteolytic enzyme in an acidic medium, followed by successive washings with water and drying in a spray dryer, allows the degradation and removal of most cellular debris from the fermented biomass, yielding a material of adequate purity for the subsequent synthesis steps.

Adding to this material an alcohol, such as methanol, ethanol, isopropanol or n-propanol, in proportions between 3 and 10 times the stoichiometric ratio of synthesis of the respective ester, and an acid catalyst, such as HCl, H2SO4 or organic tin salts, in particular butyl stanoic acid (Fascat 4100), the polymeric chain of PHB-co-HV is broken, forming the respective esters. Subsequently, by alkalizing these esters, the mixture of (R)-3-hydroxybutyrate and (R)-3-hydroxyvalerate is achieved.

The invention described here, which is a process for synthesizing the mixture of these two components to be used for nutritional and therapeutic purposes, assumes an extremely relevant aspect: first, it leads to the production of optically active (R)-3-hydroxybutyrate and (R)-3-hydroxyvalerate in high purity, which are metabolically more efficient than a mixture of racemic components. Secondly and more importantly, the assimilation of (R)-3-hydroxybutyrate as an energy source is complemented by the anaplerotic component (R)-3-hydroxyvalerate, which can restore the levels of fundamental intermediates of the TCA cycle. Furthermore, unlike triheptanoin, which is transported to the interior of the cell only by diffusion, (R)-3-hydroxyvalerate has a specific and active transporter, resulting in faster and more efficient migration across the cell membrane.

The present disclosure provides a combination of (D)-β-hydroxybutyric acid (“D-BHB”), (D)-β-hydroxyvaleric acid (“D-BHV”), and (D)-1,3 butanediol (“D-1,3BD”). Numerous non-limiting examples providing exemplary proportions of these compounds are provided. These combinations and compositions containing such combinations may be prepared as food and beverage products for human consumption, thereby providing a dietary source of exogenous ketones. The resulting mixture can exhibit reduced acidity, better flavoring, and reduce or avoid the need to add additional salts to the composition to improve palatability. Moreover, the combinations disclosed herein can have a greater efficacy than other exogenous ketone compositions. When administered to a subject, the disclosed combinations of D-BHB and D-1,3BD and the disclosed combinations of D-BHB, D-BHV, and D-1,3BD exhibit an increase in blood ketones that is greater than administration of either constituent individually. Thus, the disclosed examples permit achieving nutritional and therapeutic benefits of sufficiently high circulating ketone bodies using less material than would otherwise be required.

In one aspect, the present disclosure provides a composition having 25% to 85% by weight of a mixture of (D)-β-hydroxybutyric acid and (D)-β-hydroxyvaleric acid and 15% to 75% by weight of (D)-1,3 butanediol. In one aspect, the molar ratio of (D)-β-hydroxybutyric acid to (D)-β-hydroxyvaleric acid may be between 1.0 to 0.01 and 0.7 to 0.3. In one aspect, the present disclosure provides a composition having 25% to 75% by molar of a mixture of (D)-β-hydroxybutyric acid and (D)-β-hydroxyvaleric acid and 25% to 75% by weight of (D)-1,3 butanediol. In one aspect, the present disclosure provides a composition having 35% to 70% by weight of a mixture of (D)-β-hydroxybutyric acid and (D)-β-hydroxyvaleric acid and 30% to 65% by weight of (D)-1,3 butanediol. In one aspect, the present disclosure provides a composition having 45% to 55% by weight of a mixture of (D)-β-hydroxybutyric acid and (D)-β-hydroxyvaleric acid and 55% to 45% by weight of (D)-1,3 butanediol. In one aspect, the present disclosure provides a composition having 48% to 52% by weight of a mixture of (D)-β-hydroxybutyric acid and (D)-β-hydroxyvaleric acid and 48% to 52% by weight of (D)-1,3 butanediol.

In one aspect, a composition, beverage or food product of the present disclosure may contain at least 5, 10, 20, 30, 40, 50, 60, 70, 80, or 90% of the D)-β-hydroxybutyric acid, the (D)-β-hydroxyvaleric acid, or both, in the form of a magnesium salt, potassium salt, calcium salt, sodium salt, or combination thereof. In some aspects, the composition, beverage or food product of the present disclosure may contain up to 50% of the D)-βf-hydroxybutyric acid, the (D)-β-hydroxyvaleric acid, or both, in the form of a magnesium or potassium salt. In some aspects, the composition, beverage or food product of the present disclosure may contain up to 30% of the D)-β-hydroxybutyric acid, the (D)-β-hydroxyvaleric acid, or both, in the form of a calcium salt. In some aspects, the composition, beverage or food product of the present disclosure may contain up to 60% of the D)-β-hydroxybutyric acid, the (D)-β-hydroxyvaleric acid, or both, in the form of a combination of magnesium, potassium, and/or calcium salts.

In another aspect, the present disclosure provides a method and composition for inducing a D-BHB plasma level increase of at least 1.4 mM within 2 hours by administering the compositions of the present disclosure.

In another aspect, the present disclosure provides a method and composition for inducing a D-BHB plasma level increase of at least 1.5 mM within 2 hours by administering the compositions of the present disclosure.

In another aspect, the present disclosure provides a method and composition for inducing a D-BHB plasma level increase of at least 2.0 mM within 2 hours by administering the compositions of the present disclosure.

In another aspect, the present disclosure provides a method and composition for maintaining a D-BHB plasma level increase of at least 1.4 mM for 2.5 hours by administering the compositions of the present disclosure.

In another aspect, the present disclosure provides a method and composition for maintaining a D-BHB plasma level increase of at least 1.4 mM for 3 hours by administering the compositions of the present disclosure.

In another aspect, the present disclosure provides a method and composition for maintaining a D-BHB plasma level increase of at least 1.4 mM for 3.5 hours by administering the compositions of the present disclosure.

In another aspect, the present disclosure provides a method and composition for maintaining a D-BHB plasma level increase of at least 1.4, 1.5, 1.6, 1.7, 1.8, 1.9, or 2 mM for 2, 2.5, 3, 3.5, or 4 hours by administering the compositions of the present disclosure.

Preferred examples include administration of a combination of D-BHB, D-BHV, and D-1,3BD in a therapeutically effective amount such that the rate change of circulating ketones in the blood of a human subject at rest is faster than the administration of an equivalent amount of either D-BHB or D-1,3BD administered alone. In one example, this includes the administration of a composition including a mixture of 43.2%% D-BHB, 1.8% D-BHV and 55% D-1,3BD. In another example, this includes the administration of a composition including a mixture of 52.8% DBHB, 2.2% D-BHV, and 45% D-1,3BD. In another example, this includes the administration of at composition including a mixture of 62.4% D-BHB, 2.6% D-BHV, and 35% D-1,3BD.

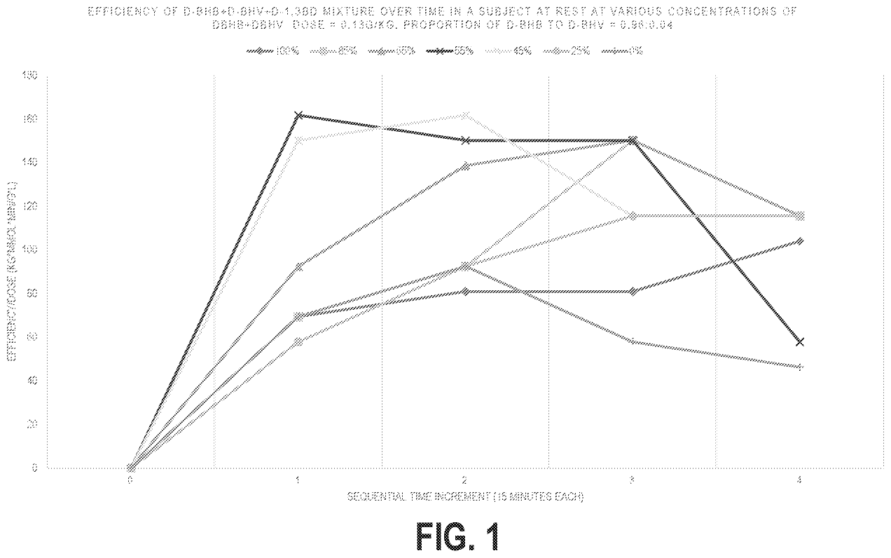

FIG. 1 shows the results of administration of D-BHB, D-BHV, and D-1,3BD over the course of a 60-minute period in various ratios in a subject at rest. Examples include using a composition including D-BHB, D-BHV, and D-1,3BD in a subject at rest. The ketotic efficiency represents the net elevation of circulating ketones a ketone supplement affects over time in an individual. Ketotic efficiency may be represented as:

$${\sum }_{t = 0}^{n} ( m Mol / L ) / ( g / kg ) ⋆ t$$

where mMol/L is the concentration of circulating p-hydroxybutyrate in the blood, g is the total dose of the combined D-BHB, D-BHV, and D-1,3,BD in grams, kg is the mass of the individual in kilograms, and t is an increment of time in minutes.

The ketotic efficiency of the D-BHB, D-BHV, and D-1,3BD blend measured over of one hour in this exemplary study is 43% to 104% greater than the administration of pure (D)-1,3 butanediol or pure (D)-β-hydroxybutyric acid alone, respectively. While this example pertains to a fasted subject at rest, the relative utility of blended vs pure constituents has also been observed in fed and active subjects.

As illustrated by the examples of FIG. 1, these exemplary compositions can advantageously elevate circulating ketones by approximately two times greater than other known compositions. Such a composition can sustain ketonemia for longer than either D-BHB or D-1,3BD can when ingested alone by a subject of a given weight as shown by the 100% line (i.e., contains D-BHB, but not D-1,3BD) and the 0% line (i.e., contains only D-1,3BD, but not D-BHB).

As shown in the following table, the increase in circulating ketone level with a 10 g dose in three human subjects at rest was maximal at between 45-55% D-BHB in the tested compositions.

| % D-BHB | Max change |
|---|---|
| Acid | in mMol/L |
| 100% | 0.9 |
| 85% | 1.3 |
| 65% | 1.3 |
| 55% | 1.4 |
| 45% | 1.4 |
| 25% | 1 |
| 0% | 0.8 |

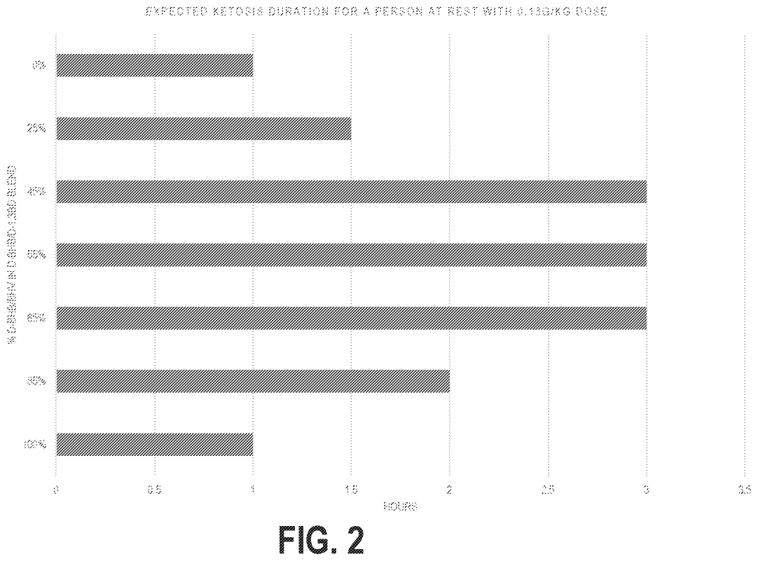

FIG. 2 represents the expected ketosis for a person at rest with a 0.13 g/kg dose of (D)-β-hydroxybutyric acid, (D)-β-hydroxyvaleric acid, and (D)-1,3 butanediol over time for the periods shown. For instance, based on the measured plasma levels illustrated in FIG. 1, it can be concluded that administration of 100% D-1,3BD does not cause a prolonged rise of blood ketone levels. A similar result was obtained upon administration of 100% D-BHB. Administration of examples in accordance with the present disclosure are expected to cause efficient, prolonged rises in circulating blood ketone levels in a manner that is unexpected based on the current literature. As shown in the following table, the highest efficiency at both 1 and 2 hours post-administration was obtained at 45% D-BHB+D-BHV (0.96:0.04) to 55% D-1,3BD and the lowest efficiency was observed when administering D-BHB or D-1,3BD alone.

| Concentration of | | |
|---|---|---|
| DBHB + DBHV | | |
| in a compositing | 1 hourketotic | 2 hours ketotic |
| containing DBHB + | efficiency | efficiency |
| DBHV and D-1,3BD | (kg*Mmol*min/g*L) | (kg*Mmol*min/g*L) |
| 100% | 335 | 566 |
| 85% | 427 | 739 |
| 65% | 497 | 780 |
| 55% | 520 | 774 |
| 45% | 543 | 826 |
| 25% | 381 | 739 |
| 0% | 266 | 462 |

As demonstrated herein, the combination of the present disclosure safely induces ketosis more rapidly than previously thought possible. For example, U.S. Pat. No. 9,138,420 shows that a peak concentration of blood concentrations of (D)-β-hydroxybutyrate produced by consuming a combination of (D/L)-β-hydroxybutyrate salt and MCT (medium chain triglycerides) oil required up to 3 hours.

The present disclosure includes mixing D-BHB, D-BHV, and D-1,3BD in a food or beverage product. For example, any of the compositions according to the examples, discussed earlier, may be included within a beverage or food product. Still further examples include the administration of the disclosed compositions of D-BHB, D-BHV, and D-1,3BD as a nutritional supplement to induce ketonemia. Still further examples include the administration of the disclosed compositions including D-BHB, D-BHV, and D-1,3BD as a nutritional supplement for the treatment of metabolic disorders, particularly those involving brain energy deficit from reduced glucose absorption capacity and aneplerotic deficiency, such as insulin resistance, glucose transporter 1 deficiency, diabetes, and central nervous system disorders, like Huntington's disease, pyruvate carboxylase deficiency, Alzheimer's disease, Parkinson's disease, and epilepsy.

An example of a method includes administering a composition to a human subject in a beverage or food product. In one aspect, the beverage or food product may be designed to be consumed in one sitting rather than over a prolonged period. The exemplary compositions described in the non-limiting examples and other disclosures provided herein may be used in such beverage or food products.

In some aspects, the present disclosure involves a unit dosage containing about 5 grams or more of the combination of D-BHB, D-BHV, and D-1,3BD of the present disclosure. In some aspects, the present disclosure involves a unit dosage containing 5-50, 6 to 49, 7 to 48, 8 to 47, 8 to 46, 9 to 45, 10 to 44, 11 to 43, 12 to 42, 13 to 40, 14 to 35, 15 to 30, 16-25, or 18 to 22 grams of the combination of D-BHB, D-BHV, and D-1,3BD of the present disclosure. In some aspects, the present disclosure includes a composition or method for inducing and maintaining ketonemia or ketosis by ingesting at least 10 grams of the combination of D-BHB, D-BHV, and D-1,3BD of the present disclosure daily (e.g., 1, 2, 3, 4, 5, 6, or more times per day). In some aspects, the present disclosure includes a composition or method for inducing and maintaining ketonemia or ketosis by ingesting at least 10 grams of the combination of D-BHB, D-BHV, and D-1,3BD of the present disclosure weekly (e.g., 5, 6, 7, 8, 9, 10, 12, 14, 21, 28 or more times per week). In some aspects, the present disclosure includes a composition or method for inducing and maintaining ketonemia or ketosis by ingesting at least 10 grams of the combination of D-BHB, D-BHV, and D-1,3BD of the present disclosure every 2, 3, 4, or 5 hours. In some aspects, the present disclosure includes a composition or method for inducing and maintaining ketonemia or ketosis by ingesting at least 10 grams of the combination of D-BHB, D-BHV, and D-1,3BD of the present disclosure at each meal. In some aspects, the present disclosure includes a composition or method for inducing and maintaining ketonemia or ketosis by ingesting at least 10 grams of the combination of D-BHB, D-BHV, and D-1,3BD of the present disclosure before, during, or after a fasted state of at least 6, 8, 10, or 12 hours.

The present disclose includes a total daily dosage of 50, 60, 70, 80, 90, 100, 110, 120, 130, 140, 150, 160, 170, 180, 190, or 200 grams of the combination of D-BHB, D-BHV, and D-1,3BD of the present disclosure.

In some aspects, the combination of D-BHB, D-BHV, and D-1,3BD of the present disclosure is administered at a dosage of 0.10 to 1 g/kg, 0.12 to 0.8 g/kg, 0.13 to 0.7 g/kg, 0.14 to 0.6 g/kg, 0.15 to 0.5 g/kg per unit dosage. In some aspects, the combination of D-BHB, D-BHV, and D-1,3BD of the present disclosure is administered at a dosage of 0.30 to 3 g/kg, 0.4 to 2.5 g/kg, 0.5 to 2 g/kg, 0.75 to 1.5 g/kg, 0.8 to 1 g/kg per day.

In some aspects, the present disclosure includes compositions for reducing or avoiding side effects such as acidosis and gastrointestinal distress upon ingestion of D-BHB, D-BHV, and D-1,3BD.

In some aspects, the present disclosure includes compositions for reducing or avoiding side effects such as intoxication from 1,3-butanediol by use of the combination of D-BHB, D-BHV, and D-1,3BD of the present disclosure.

According to other methods, a composition including approximately 15% to approximately 85% D-BHB, between substantially no D-BHV to approximately 26% D-BHV, and between approximately 15% to approximately 75% D-1,3BD, such as any of the compositions set forth in the examples (or variants thereof) is administered to a human subject to increase levels of circulating ketones in the blood of the subject. In such compositions, the proportion of D-BHB to D-BHV can be between 1.0 to 0 and 0.7 to 0.3, 0.99 to 0.01, 0.98 to 0.02, 0.97 to 0.03, 0.96 to 0.04, 0.95 to 0.05, 0.9 to 0.1, 0.85 to 0.15, 0.8 to 0.2, or 0.75 to 0.25. Such compositions may be administered using specific carriers, e.g., as described below and illustrated in several examples.

According to another example, the individual constituent components of approximately 15% to approximately 85% D-BHB, between substantially no D-BHV to approximately 26% D-BHV, and between approximately 15% to approximately 75% D-1,3BD may be taken in rapid succession, such that, for example, D-BHB is taken first, D-BHV is taken second, and D-1,3,BD is taken third. According to one example, the D-BHB and D-BHV may be taken first, and the D-1,3BD may be taken second. The D-BHV and D-BHB may be taken simultaneously as a single mixture of these compounds in appropriate amounts. In another example, the D-1,3BD may be taken first, and the D-BHB and D-BHV may be taken second. In other examples, one portion of the composition may be taken 2, 3, or 5 minutes before the other portion of the composition.

In one aspect, the present disclosure involves the described compounds, i.e., (D)-β-hydroxybutyric acid, (D)-β-hydroxyvaleric acid, and/or (D)-1,3 butanediol, that are not in the form of a salt (e.g., not a sodium, magnesium, calcium and/or potassium salt). In one aspect, the present disclosure involves the described compounds, i.e., (D)-β-hydroxybutyric acid, (D)-β-hydroxyvaleric acid, and/or (D)-1,3 butanediol, that are not in the form of an ester. In one aspect, the present disclosure involves compositions comprising (D)-β-hydroxybutyric acid, (D)-β-hydroxyvaleric acid, and/or (D)-1,3 butanediol in a buffer-free composition. In one aspect, the present disclosure involves compositions comprising (D)-β-hydroxybutyric acid, (D)-β-hydroxyvaleric acid, and/or (D)-1,3 butanediol in a lactose-free composition. In one aspect, the present disclosure involves compositions comprising (D)-β-hydroxybutyric acid, (D)-β-hydroxyvaleric acid, and/or (D)-1,3 butanediol in a gluten-free composition. In one aspect, the present disclosure involves compositions comprising (D)-β-hydroxybutyric acid, (D)-β-hydroxyvaleric acid, and/or (D)-1,3 butanediol in a soy-free composition. In one aspect, the present disclosure involves compositions comprising (D)-β-hydroxybutyric acid, (D)-β-hydroxyvaleric acid, and/or (D)-1,3 butanediol in a caffeine-free composition. In one aspect, the present disclosure involves compositions comprising (D)-β-hydroxybutyric acid, (D)-β-hydroxyvaleric acid, and/or (D)-1,3 butanediol in a carbohydrate-free composition. In one aspect, the present disclosure involves compositions comprising (D)-β-hydroxybutyric acid, (D)-β-hydroxyvaleric acid, and/or (D)-1,3 butanediol in a composition free of 3-hydroxybutyl-3-hydroxybutyrate. In one aspect, the present disclosure involves compositions comprising (D)-β-hydroxybutyric acid, (D)-β-hydroxyvaleric acid, and/or (D)-1,3 butanediol in a composition free of 3-hydroxybutyl-3-hydroxy-ethyl butyrate. In one aspect, the present disclosure involves compositions comprising (D)-β-hydroxybutyric acid, (D)-β-hydroxyvaleric acid, and/or (D)-1,3 butanediol in a composition free of 3-hydroxybutyl-3-hydroxy-butanoate. In one aspect, the present disclosure involves compositions comprising (D)-β-hydroxybutyric acid, (D)-β-hydroxyvaleric acid, and/or (D)-1,3 butanediol in a composition free of ketone ester. In one aspect, the present disclosure involves compositions comprising (D)-β-hydroxybutyric acid, (D)-β-hydroxyvaleric acid, and/or (D)-1,3 butanediol in a composition free of acetoacetate.

In one aspect, the present disclosure involves compositions comprising (D)-β-hydroxybutyric acid, (D)-β-hydroxyvaleric acid, and/or (D)-1,3 butanediol in a composition free of the following compound D beta hydroxybutyrate, DL 1,3-butanediol ester.

In one aspect, the present disclosure involves compositions comprising (D)-β-hydroxybutyric acid, (D)-β-hydroxyvaleric acid, and/or (D)-1,3 butanediol in a sugar-free composition. In one aspect, the present disclosure involves compositions comprising (D)-β-hydroxybutyric acid, (D)-β-hydroxyvaleric acid, and/or (D)-1,3 butanediol in a composition free of polyesters of (D)-β-hydroxybutyric acid, (D)-β-hydroxyvaleric acid, and/or (D)-1,3 butanediol. In one aspect, the present disclosure involves compositions comprising (D)-β-hydroxybutyric acid, (D)-β-hydroxyvaleric acid, and/or (D)-1,3 butanediol in a composition free of D ethyl 3-hydroxybutyrate. In one aspect, the present disclosure involves compositions comprising (D)-β-hydroxybutyric acid, (D)-β-hydroxyvaleric acid, and/or (D)-1,3 butanediol in a composition free of medium chain triglycerides. In one aspect, the present disclosure involves compositions comprising (D)-β-hydroxybutyric acid, (D)-β-hydroxyvaleric acid, and/or (D)-1,3 butanediol in a composition free of medium chain fatty acids. In one aspect, the present disclosure involves compositions comprising (D)-β-hydroxybutyric acid, (D)-β-hydroxyvaleric acid, and/or (D)-1,3 butanediol in a composition free of esters of medium chain fatty acids.

In one aspect, the present disclosure involves compositions comprising (D)-β-hydroxybutyric acid, (D)-β-hydroxyvaleric acid, and/or (D)-1,3 butanediol in combination with a sugar alcohol. In one aspect, the present disclosure involves compositions comprising (D)-β-hydroxybutyric acid, (D)-β-hydroxyvaleric acid, and/or (D)-1,3 butanediol in combination with erythritol, sorbitol, mannitol, xylitol, aspartame, stevia glycosides, allulose, monk fruit (also referred to as monk fruit extract), and similar sweeteners having zero or low calories. A sweetener can be “low-calorie”, i.e., it imparts desired sweetness when added to a sweetenable composition (such as, for example, as beverage) and has less than 40 calories per 8 oz serving. A sweetener can be “zero-calorie”, i.e., it imparts desired sweetness when added to a sweetenable composition (such as, for example, a beverage) and has less than 5 calories per 8 oz. serving, preferably 0 calories per 8 oz. serving.

Compositions based on the above examples may be mixed with a carrier comprising a food or beverage product as illustrated by the examples below. The present disclosure includes products including, but not limited to protein bars, nutritional and sports beverages, fruit juice, zero calorie iced caffeinated beverages, snacks, tea beverages, carbonated beverages, energy gels, and alcoholic beverages. The present disclosure also includes fermented foods and beverages containing the compositions described herein. The preferred compositions may be combined in foods or beverages that exhibit various nutritional criteria such as low-calorie foods and beverages for weight control, low calorie and low carbohydrate for facilitating weight control and/or weight loss, low carbohydrate and/or high fat for those following a ketogenic diet, and high carbohydrate and/or high protein for athletes.

Where a medicament or nutritional product of the invention is for use in a beverage, food, snack bar, gel or the like, it is convenient to use it in the form of a liquid or solid, preferably with a composition having approximately 15% to approximately 85% D-BHB, between substantially no D-BHV to approximately 26% D-BHV, and between approximately 15% to approximately 75% D-1,3BD, including any of the examples, disclosures or combinations thereof. The resulting compositions may be administered in a dosage of greater than about 0.02 g/kg and more preferably between about 0.1 to about 0.9 g/kg.

The present invention will now be described further by way of illustration only by reference to the following examples. Further embodiments falling within the scope of the invention will occur to those skilled in the art in light of these. Each of these examples is expected to increase blood ketone levels by about 1.5 mMol, sustained for approximately 30 minutes and tapering over the course of 3 hours. For example, a person may achieve and maintain a ketone body level of about 1.3, 1.4, 1.5, 1.6, 1.7, 1.8, 1.9, 2.0, 2.1, 2.2, 2.3, 2.4, 2.5 mM or higher according to the methods of the present disclosure.

### Example 1

A 10 L fermenter of working volume received 5 L of a 10 g/L phosphoric acid solution, 15 g/L ammonium sulfate, 1 g/L magnesium sulfate, 2 g/L propionic acid and inverted cane molasses, for a final concentration of 20 g/L reducing sugars. After autoclaving, the pH was adjusted to 6.8, then 100 mL of a cell suspension of the bacterium Alcaligenes latus DSM 1122 was added, keeping the oxygen saturation in the medium at 20% with the injection of sterile air. The pH of the medium was maintained at the value of 6.8 with the continuous addition of 2N NaOH. After about 12 hours, a molasses solution containing 600 g/L of reducing sugars and 60 g/L of propionic acid was continuously added, maintaining a concentration of reducing sugars in the fermenter around 5 g/L, for a period of 48 hours. At the end of this period, a volume of fermented material of 9 L was obtained. This material was then submitted to a heat treatment at 80 C for 15 minutes, yielding a cell suspension with about 180 g/L of dry matter containing 120 g/L of PHB-co-HV, with a molar fraction of HV of 10%.

### Example 2

The cell suspension obtained by the method described in Example 1 was subjected to a process of centrifugation and washing with a 50 mM Citrate buffer pH 4.5. The collected cell mass was resuspended in 50 mM citrate buffer pH 4.5, for a final cell concentration of 180 g/L dry basis. To this cell suspension, a solution of the protease Bromelain was added, in an amount such that the proteolytic activity evaluated in the cell suspension was 15 IU/ml. This suspension was kept at a temperature of 50 C for 12 hours, then centrifuged and washed with distilled water twice and finally dried in a spray dryer, generating approximately 1300 g of a slightly grayish powder containing PHB-co-HV with a purity of 93% on a dry basis.

### Example 3

In a vitrified reactor with a total volume of 12 L, 6 L of anhydrous ethanol, 1,000 g of PHB-co-HV, obtained as described in example 2, and 60 mL of 32% hydrochloric acid were added. This mixture was then kept in the reactor under agitation, at a temperature of 110 C, for 24 hours. At the end of this period, the reactor was ventilated to remove the ethyl ether formed during the reaction and 25 mL of a 50% NaOH solution was added, in order to obtain a pH value of 5.5. The reactor was then heated to 80° C. until all excess ethanol was removed. The suspension obtained was then subjected to vacuum distillation, at an increasing temperature between 75 and 100 C, and the vapors were collected and condensed.

At the end of the vacuum distillation process, 950 g of a viscous, colorless liquid were obtained, containing 85% of ethyl-(R)-3-hydroxybutyrate, 8% of ethyl-(R)-3-hydroxyvalerate and 4% of ethanol.

### Example 4

To 150 ml of a solution of ethyl-(R)-3-hydroxybutyrate and ethyl-(R)-3-hydroxyvalerate obtained according to example 3, 300 ml of water was added. 80 g of 50% NaOH was then added over a period of 1 h. The solution obtained was then subjected to evaporation under a vacuum of 5 mmHg absolute, at a temperature of 50 C, until there was no further significant evaporation. The solution obtained, a slightly yellowish and viscous liquid, was then evaporated in a spray dryer, yielding 110 g of a white powder. Analysis by HPLC showed that the composition of this powder was 90% (R) sodium-3-hydroxybutyrate and 9/a (R) sodium-3-hydroxyvalerate.

### Example 5

To 150 ml of a solution of ethyl-(R)-3-hydroxybutyrate and ethyl-(R)-3-hydroxyvalerate obtained according to example 3, 300 ml of water was added. 110 g of 50% KOH was then added, over a period of 1 h. The solution obtained was then subjected to evaporation under a vacuum of 5 mmHg absolute, at a temperature of 50 C, until there was no further significant evaporation. The resulting solution, a slightly yellowish and viscous liquid, was then evaporated in a spray dryer, yielding 125 g of a white powder. Analysis by HPLC showed that the composition of this powder was 90% (R) potassium-3-hydroxybutyrate and 9% (R) potassium-3-hydroxyvalerate.

### Example 6

To 150 ml of a solution of ethyl-(R)-3-hydroxybutyrate and ethyl-(R)-3-hydroxyvalerate obtained according to example 3, 300 ml of water was added. 80 g of 50% NaOH was then added over a period of 1 h. The solution obtained was then passed through a bed of cationic resin, balanced with HCl, so that all the sodium present in the original solution was replaced by H+. The resulting solution was subjected to evaporation under a vacuum of 5 mmHg absolute, at a temperature of 50 C, until there was no further significant evaporation. At the end of the evaporation, 125 mL of an acidic, colorless and viscous liquid were obtained, whose analysis by HPLC showed to be a mix of 63% (R)-3-hydroxybutyric acid, 6% of (R)-3-hydroxyvaleric acid and 30% water.

### Example 7A

A human subject suffering from a metabolic disorder involving deficiency of glucose transporter 1 (GLUT1-DS) is administered a combination of (R)-3-hydroxybutyrate and (R)-3-hydroxyvalerate or (R)-3-hydroxybutyrate, (R)-3-hydroxyvalerate, and (D)-1,3 butanediol in an amount of 0.10 to 1 g/kg, 0.12 to 0.8 g/kg, 0.13 to 0.7 g/kg, 0.14 to 0.6 g/kg, or 0.15 to 0.5 g/kg per unit dosage. In some instances, the human subject is administered the combination at a dosage of 0.30 to 3 g/kg, 0.4 to 2.5 g/kg, 0.5 to 2 g/kg, 0.75 to 1.5 g/kg, 0.8 to 1 g/kg per day. The proportion of (R)-3-hydroxybutyrate to (R)-3-hydroxyvalerate is 1.0 to 0 and 0.7 to 0.3, 0.99 to 0.01, 0.98 to 0.02, 0.97 to 0.03, 0.96 to 0.04, 0.95 to 0.05, 0.9 to 0.1, 0.85 to 0.15, 0.8 to 0.2, or 0.75 to 0.25. The human subject achieves a circulating ketone level of about 1.3, 1.4, 1.5, 1.6, 1.7, 1.8, 1.9, 2.0, 2.1, 2.2, 2.3, 2.4, 2.5 mM or higher. The human subject achieves an increase in circulating ketone level from baseline (prior to administration) of at least 0.5 mM, 0.75 mM, 1.0 mM, 1.1 mM, 1.2 mM, 1.3 mM, 1.4 mM, 1.5 mM, 1.6 mM, 1.7 mM, or higher. A unit dosage includes 5-50, 6 to 49, 7 to 48, 8 to 47, 8 to 46, 9 to 45, 10 to 44, 11 to 43, 12 to 42, 13 to 40, 14 to 35, 15 to 30, 16-25, or 18 to 22 grams of the combination. The human subject is administered at least 10 grams of the combination daily or multiple times per day (e.g., 1, 2, 3, 4, 5, 6, or more times per day). The human subject is administered at least 5 grams of the combination every 2, 3, 4, or 5 hours.

### Example 7B

A human subject suffering from at least one of Huntington's disease, Parkinson's disease, Alzheimer's disease, senile dementia, Pick's disease, and Cretzfeldt-Jacobs' disease is administered a combination of (R)-3-hydroxybutyrate and (R)-3-hydroxyvalerate or (R)-3-hydroxybutyrate, (R)-3-hydroxyvalerate, and (D)-1,3 butanediol in an amount of 0.10 to 1 g/kg, 0.12 to 0.8 g/kg, 0.13 to 0.7 g/kg, 0.14 to 0.6 g/kg, or 0.15 to 0.5 g/kg per unit dosage. In some instances, the human subject is administered the combination at a dosage of 0.30 to 3 g/kg, 0.4 to 2.5 g/kg, 0.5 to 2 g/kg, 0.75 to 1.5 g/kg, 0.8 to 1 g/kg per day. The proportion of (R)-3-hydroxybutyrate to (R)-3-hydroxyvalerate is 1.0 to 0 and 0.7 to 0.3, 0.99 to 0.01, 0.98 to 0.02, 0.97 to 0.03, 0.96 to 0.04, 0.95 to 0.05, 0.9 to 0.1, 0.85 to 0.15, 0.8 to 0.2, or 0.75 to 0.25. The human subject achieves a circulating ketone level of about 1.3, 1.4, 1.5, 1.6, 1.7, 1.8, 1.9, 2.0, 2.1, 2.2, 2.3, 2.4, 2.5 mM or higher. The human subject achieves an increase in circulating ketone level from baseline (prior to administration) of at least 0.5 mM, 0.75 mM, 1.0 mM, 1.1 mM, 1.2 mM, 1.3 mM, 1.4 mM, 1.5 mM, 1.6 mM, 1.7 mM, or higher. A unit dosage includes 5-50, 6 to 49, 7 to 48, 8 to 47, 8 to 46, 9 to 45, 10 to 44, 11 to 43, 12 to 42, 13 to 40, 14 to 35, 15 to 30, 16-25, or 18 to 22 grams of the combination. The human subject is administered at least 10 grams of the combination daily or multiple times per day (e.g., 1, 2, 3, 4, 5, 6, or more times per day). The human subject is administered at least 5 grams of the combination every 2, 3, 4, or 5 hours.

### Example 7C

A human subject suffering from epilepsy is administered a combination of (R)-3-hydroxybutyrate and (R)-3-hydroxyvalerate or (R)-3-hydroxybutyrate, (R)-3-hydroxyvalerate, and (D)-1,3 butanediol in an amount of 0.10 to 1 g/kg, 0.12 to 0.8 g/kg, 0.13 to 0.7 g/kg, 0.14 to 0.6 g/kg, or 0.15 to 0.5 g/kg per unit dosage. In some instances, the human subject is administered the combination at a dosage of 0.30 to 3 g/kg, 0.4 to 2.5 g/kg, 0.5 to 2 g/kg, 0.75 to 1.5 g/kg, 0.8 to 1 g/kg per day. The proportion of (R)-3-hydroxybutyrate to (R)-3-hydroxyvalerate is 1.0 to 0 and 0.7 to 0.3, 0.99 to 0.01, 0.98 to 0.02, 0.97 to 0.03, 0.96 to 0.04, 0.95 to 0.05, 0.9 to 0.1, 0.85 to 0.15, 0.8 to 0.2, or 0.75 to 0.25. The human subject achieves a circulating ketone level of about 1.3, 1.4, 1.5, 1.6, 1.7, 1.8, 1.9, 2.0, 2.1, 2.2, 2.3, 2.4, 2.5 mM or higher. The human subject achieves an increase in circulating ketone level from baseline (prior to administration) of at least 0.5 mM, 0.75 mM, 1.0 mM, 1.1 mM, 1.2 mM, 1.3 mM, 1.4 mM, 1.5 mM, 1.6 mM, 1.7 mM, or higher. A unit dosage includes 5-50, 6 to 49, 7 to 48, 8 to 47, 8 to 46, 9 to 45, 10 to 44, 11 to 43, 12 to 42, 13 to 40, 14 to 35, 15 to 30, 16-25, or 18 to 22 grams of the combination. The human subject is administered at least 10 grams of the combination daily or multiple times per day (e.g., 1, 2, 3, 4, 5, 6, or more times per day). The human subject is administered at least 5 grams every 2, 3, 4, or 5 hours.

### Example 7D

A human subject suffering from a metabolic disorder involving deficiency of the enzyme pyruvate carboxylase (PC) is administered a combination of (R)-3-hydroxybutyrate and (R)-3-hydroxyvalerate or (R)-3-hydroxybutyrate, (R)-3-hydroxyvalerate, and (D)-1,3 butanediol in an amount of 0.10 to 1 g/kg, 0.12 to 0.8 g/kg, 0.13 to 0.7 g/kg, 0.14 to 0.6 g/kg, or 0.15 to 0.5 g/kg per unit dosage. In some instances, the human subject is administered the combination at a dosage of 0.30 to 3 g/kg, 0.4 to 2.5 g/kg, 0.5 to 2 g/kg, 0.75 to 1.5 g/kg, 0.8 to 1 g/kg per day. The proportion of (R)-3-hydroxybutyrate to (R)-3-hydroxyvalerate is 1.0 to 0 and 0.7 to 0.3, 0.99 to 0.01, 0.98 to 0.02, 0.97 to 0.03, 0.96 to 0.04, 0.95 to 0.05, 0.9 to 0.1, 0.85 to 0.15, 0.8 to 0.2, or 0.75 to 0.25. The human subject achieves a circulating ketone level of about 1.3, 1.4, 1.5, 1.6, 1.7, 1.8, 1.9, 2.0, 2.1, 2.2, 2.3, 2.4, 2.5 mM or higher. The human subject achieves an increase in circulating ketone level from baseline (prior to administration) of at least 0.5 mM, 0.75 mM, 1.0 mM, 1.1 mM, 1.2 mM, 1.3 mM, 1.4 mM, 1.5 mM, 1.6 mM, 1.7 mM, or higher. A unit dosage includes 5-50, 6 to 49, 7 to 48, 8 to 47, 8 to 46, 9 to 45, 10 to 44, 11 to 43, 12 to 42, 13 to 40, 14 to 35, 15 to 30, 16-25, or 18 to 22 grams of the combination. The human subject is administered at least 10 grams of the combination daily or multiple times per day (e.g., 1, 2, 3, 4, 5, 6, or more times per day). The human subject is administered at least 5 grams every 2, 3, 4, or 5 hours.

### Example 8

Brain pattern changes in human subjects were measured after consumption of optically active mixtures of (R)-3-hydroxybutyrate, (D)-1,3 butanediol, and (R)-3-hydroxyvalerate.

### Data Acquisition Specifications

1. QEEG Acquisition. Human electroencephalogram (EEG) study was recorded from 19 electrodes, according to IFCN Standards placed using the International 10/20 placement system referenced in Linked Ears Montage, recorded for eight (8) minutes total duration in Eyes Open and eight (8) minutes total duration in Eyes Closed conditions in accordance with the International 10/20 Electrode Placement System, Edit length includes one (1) minute of EEG with test-retest reliability and split-half reliability of greater than ninety-five percent. All electrode impedances below 10,000 Ohms. The EEG amplifiers have a bandpass from 0.5 to 70 Hz (3 dB points), with a 60 Hz notch filter. Data sampled at a rate of 200 Hz with 12-bit resolution. Patients monitored for vigilance during EEG recording.
2. Basic demographic intake: including Covid19 history/status (if residual symptoms, characterize as mild, moderate, or severe).
3. Finger Prick test (assay) of serum ketone status. Keto Mojo beta-ketone & blood glucose plus hematocrit & hemoglobin monitoring system.
4. Self-report scales: PQ-9 (general health), age, weight, history of fasting/carbohydrate restricted diet.

### Subjects

1. Inclusion Criteria: Ten (10) Male and Seven (7) Females subjects between ages of twenty (23) to sixty-five (65) years old.

| | Study | Female | Male | Study | Female | Male |
|---|---|---|---|---|---|---|
| | Average | Avg | Avg | Median | Median | Median |
| | Age | Age | Age | Age | Age | Age |
| Age | 41.8 | 41.6 | 41.3 | 44.5 | 43 | 43 |

2. Exclusion Criteria: Active substance abuse, Covid infection sixty (60) days prior; any infection or medical conditions active at time of measurement, anyone following a strict ketogenic diet or low carbohydrate intake.

### Study Process

Data collection day zero (0):
   1. Participants in a rested (no exercise in previous four (4) hours) and semi-fasted (no food in previous four (4) hours) state.
   2. Baseline QEEG taken after confirming >/=0.3 Mmol/L ketone levels in the bloodstream.
   3. Resultant QEEG measurement taken approximately thirty (30) minutes after orally consuming (drinking) 237 ml of optically active mixtures of (R)-3-hydroxybutyrate, (D)-1,3 butanediol, and (R)-3-hydroxyvalerate and confirmed elevated ketones of </=1.0 Mmol/L in the bloodstream.

| | Minute Zero > / = 0.3 | PK Minute Thirty < / = 1.0 |
|---|---|---|
| | Mmol/L | Mmol/L |
| Mmol/L Avg | 0.138888889 | 1.038888889 |

### Data Goals

1. Measure Brain Pattern Change: Determine functional brain pattern change as characterized using an electroencephalogram scan.
2. Quantify Effect on Brainwaves: Analyze the change in Delta, Theta, Alpha, Beta and Gamma brainwaves.

### Measurement and Analysis Methods

Quantitative EEG (QEEG) The EEG was obtained from 19 standard locations over the scalp according to the 10-20 system: Fp1, Fp2, F3, F4, F7, F8, T3, T4, C3, C4, P3, P4, T5, T6, O1, O2, Fz, Cz, and Pz. Discoidal EEG tin electrodes were fixed, after a careful cleaning of the skin, using a conductor paste, and connected to the input box of the digital Electroencephalographic system Medicid-05 (Neuronic, S.A.). Monopolar leads were recorded, using linked ears as a reference. Technical parameters for EEG were gain 20,000, pass-band filters 0.1-70 Hz, “notch” filter at 60 Hz, the noise level of 2 μV (root mean square), sampling frequency 200 Hz, and electrode-skin impedance never higher than 5 KΩ. A bipolar chest electrocardiogram lead (ECG), for monitoring purposes, was simultaneously recorded with the EEG, with filters selected from 0.5 to 30 Hz.
EEG visual inspection and selection of samples for quantitative EEG (QEEG): Two experts visually inspected the recorded QEEGs, and discharged the noised segments with movements or other biological artifacts EEG segments of no less than 65 seconds from the non-contaminated records were selected for each experimental section, which were later exported to an ASCII file, using the own facilities of the MEDICID-05 system, containing a matrix of the original EEG values, corresponding to the segments selected by the specialists, for offline ulterior processing. Three ASCII files containing the EEG information corresponding to each subject in the different experimental conditions were created and stored for further quantitative analysis.
QEEG analysis: EEG samples from every experimental section contained in the ASCII files previously described were imported by a specifically tailored software tool developed with Matlab version 7.10.0.499 R2010a (The Mathworks, Inc.). This program included different actions, including pre-processing of EEG samples, estimation of the power spectral densities (PSD) for every EEG lead; computation of different spectral indices, and finally, an output of these results to a database developed with Microsoft Access to specifically dedicated files.
EEG pre-processing: The EEG values of every one of the 19 leads were submitted to a previous pre-processing set of actions consisting in: (a) subtraction of the mean value of the sequence of EEG values to diminish the effect of the D.C. component of the time series; (b) application of a median filter to exclude outliers or abnormally high amplitude values; (c) standard linear detrending to avoid any possible drifts in the series; (d) high-pass digital filtering (low cutoff frequency of 0.5 Hz); (e) lowpass digital filtering (high cutoff frequency of 55 Hz) using a six order Butterworth filter.
Computation of the PSD and the spectral indices: The first 12,288 samples of the EEG values of each EEG lead were submitted to a spectral analysis implemented with the Welch periodogram method with a Hann window to avoid as possible the leakage effect. Windows of 1024 samples (5.12 s), overlapped every 512 samples, were used by this algorithm yielding 23 consecutive windows, and the results of the estimated PSD for every discrete spectral frequency were averaged to obtain the global smoothed spectrum for each EEG lead. The spectral resolution for this process was 1/5.12 s or approximately 0.195 Hz. The first six discrete frequencies, including the D.C. or zero frequency, were discarded, and for the measurements of the PSD, the values of the discrete frequencies were submitted to integration within limits selected for the different EEG bands: Delta band (1.17-3.5 Hz), Theta (3.5-7.5 Hz), Alpha (7.5-11 Hz), Beta (15-25 Hz), and Gamma (25-55 Hz). The numbers of discrete frequencies obtained for the calculations of the PSD for each band were: Delta band (12), Theta (21), Alpha (18), Sigma (20), Beta (51), and Gamma (154). The PSD for the EEG in each band was also measured in normalized units using the standard procedure, calculating the percent of the PSD concerning the total PSD in the whole investigated spectral range. As previously described, the program automatically exported all the results to a database and dedicated files for posterior storing and graphical and statistical analysis of the processed information. The description of the complete methodology for QEEG records and analysis can be found elsewhere.
Statistical Analysis: Statistical analysis was performed using the program “Neuronic Statistics” from Neuronic S.A. QEEG bands comparing Baseline and after Ketone Slixir intaked, were calculated by using Mann-Whitney tests or t-tests of independent samples for continuous variables, and chi-square or Fisher's exact tests for dichotomous variables. Grand averages of the QEEG variables for all subjects will be calculated before and after taking the mixture.

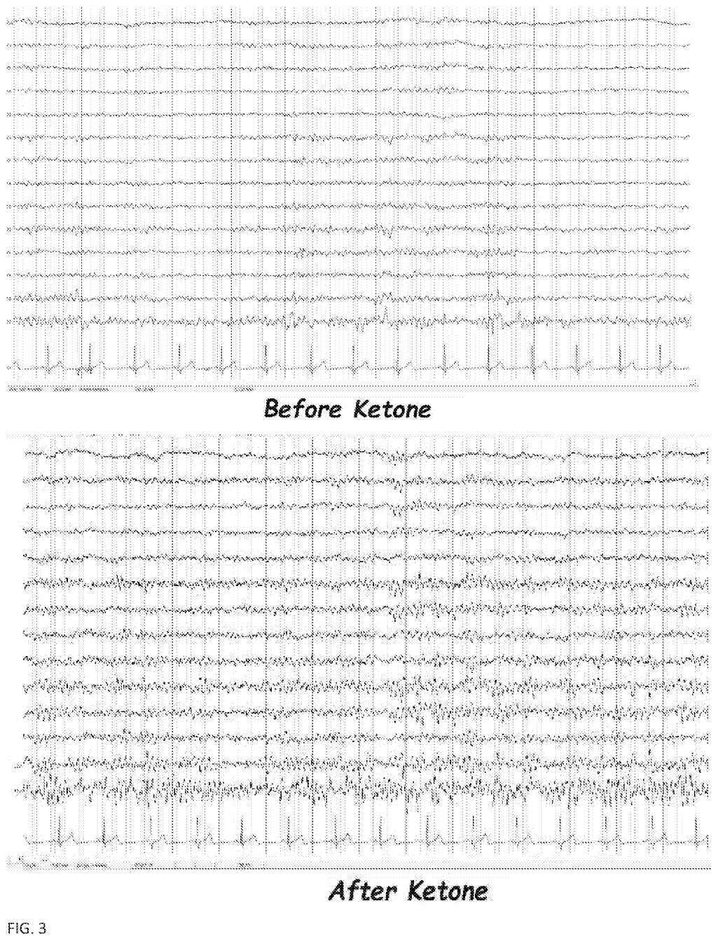

FIG. 3 is an electroencephalogram (EEG) of a female human test subject aged 46 before administration of the mixture (top) and after administration of the mixture (bottom). Better modulation of the bioelectrical activity was observed after administration. Alpha amplitude and its waxing and waning are enhanced after administration. Note, that in the raw data without quantitative analysis, it is not easy to visualize changes in delta and gamma bands.

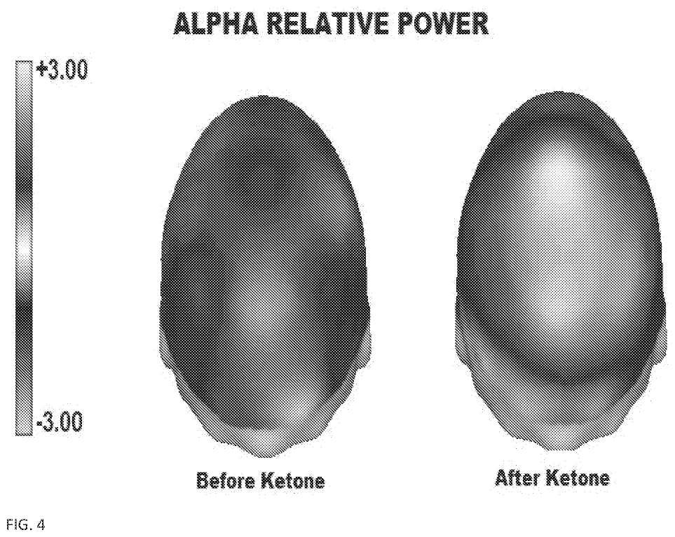

FIG. 4 shows that there is a significant augmentation of the Alpha Relative Power comparing baseline record and after consuming the mixture. There is a significant increase of the Alpha Relative Power (with p value of <0.01%), shown in FIG. 4. After exogenous ketone mixture consumption, there was a better modulation of the Alpha rhythm in the raw background activity as well. The light/white regions in the center of the brain after ketone consumption correspond to +3.00 on the Alpha Relative Power scale. See below table for qualitative summary.

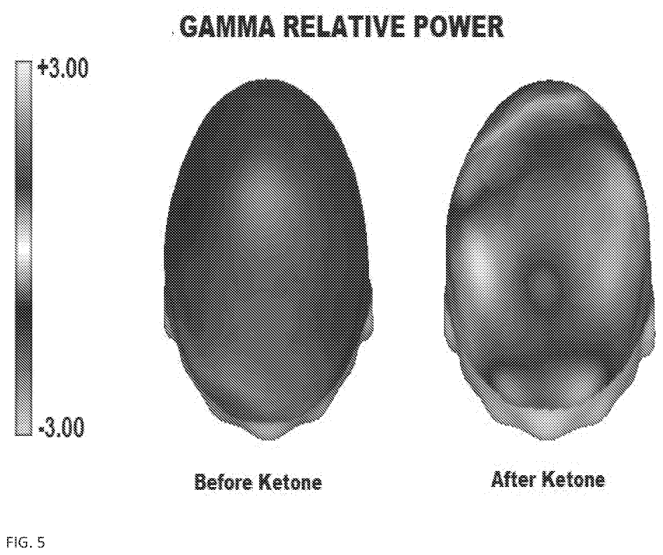

FIG. 5 shows that there is a significant augmentation of the Gamma Relative Power comparing baseline record and after consuming the mixture. After exogenous ketone mixture consumption, there is a significant increase of the Gamma Relative Power (with p value of <0.01%). Note that this increment is greater in the parietal regions, probably indicating an improvement of cognitive functions as shown in FIG. 5. See below table for qualitative summary.

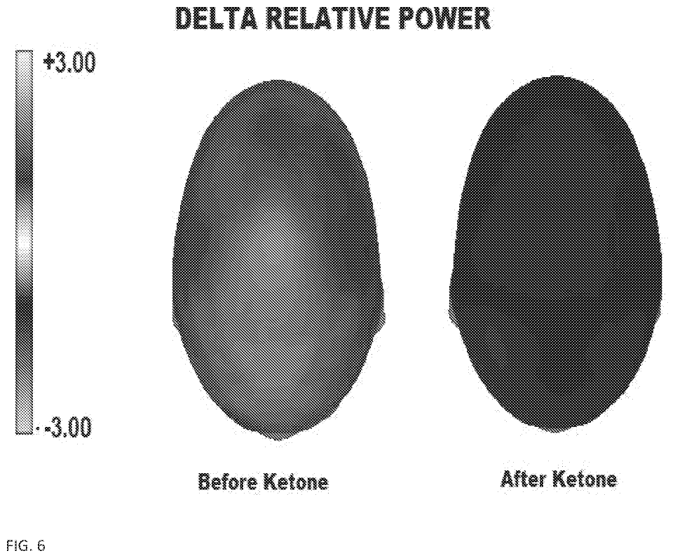

FIG. 6 shows that there is a significant decrement of the Delta Relative Power comparing baseline record and after consuming the mixture with p value of <0.01%. See below table for qualitative summary.

| | Minute Zero | Minute 30 | Change in |
|---|---|---|---|
| | Baseline QEEG | Resultant QEEG | Relative |
| Brainwave | Scan | Scan | Power |
| Alpha Relative Power | 15.75 | 18.57 | +17.90% |
| Gamma Relative | 10.77 | 12.52 | +16.25% |
| Power | | | |
| Delta Relative Power | 14.76 | 13.82 | −6.37% |

Mean standard deviations of Relative Powers of study participants before and after consumption of the mixture.

Alpha waves (8-12 Hz)—Alpha waves are predominantly associated with a relaxed, alert, and focused state. When an individual's alpha is within normal ranges, there is a sense of calmness, and the individual tends to experience good moods. Alpha is a common state for the brain and occurs whenever a person is alert (it is a marker for alertness and sleep), but not actively processing information. An individual is highly focused to execute. They are strongest over the occipital (back of head) cortex and over the frontal cortex. Significantly increases alpha relative and improves focus, flow state, learning and creativity.

Gamma waves (38-80 Hz)—Gamma waves originate in the thalamus and move from the back of the brain to the front and back again 40 times per second in a rapid “full sweep” action. This makes the gamma state one of peak mental and physical performance. Gamma is the brainwave state of being “in the zone.” Gamma brain waves are associated with the “feeling of blessings” reported by experienced meditators, such as monks and nuns. Gamma waves are associated with peak concentration, high levels of cognitive functioning, increased mental processing, happiness, better perception of reality, incredible focus, better self-control, and richer sensory experience.

Delta waves (0.5-4 HZ)—Delta waves are often referred to as “slow waves.” These waves are associated with a brain network at rest. When brain cells are resting, they are restoring their supply of neurotransmitters, repairing, and strengthening pathways of memory and learning. Delta waves are the predominant waves in deep sleep. Abnormal delta waves are seen in brain injury, coma, and seizures as well as many other conditions. Too much delta can indicate a brain that is ‘asleep’. A decrease in delta relative power waves indicates a heightened state of alertness and energy. There was significant augmentation amongst the participants in the study to the relative power of Alpha brainwaves and Gamma brainwaves and dec as compared to the baseline data. In addition to the significant decrease to the relative power of Delta brainwaves the brain is in harmony for a heightened state of alertness, focus, energy, and cognition.

The data produced in this study lend credence and further advance the established and extensive scientific literature that characterizes how the ketotic state improves brain function. More specifically, in this analysis of subjects who consumed the mixture and underwent functional brain mapping known as QEEG, statistically robust changes were seen that correlate with a functionally better neurophysiologic profile. In this cohort of healthy normals, it was demonstrated that the exogenous ketone mixture could reliably raise serum ketone levels, and while in this metabolically enhanced state. QEEG was shown to improve across three important bioelectrical markers that reflect an optimized brain state. Namely, the analysis of the brain's electrical activity the metrics indicate significant improvement in bioelectrical activity generation after consuming the mixture. Amongst the participants in the study there was significant augmentation of relative power of Alpha brainwaves and Gamma brainwaves as compared to the baseline data. There was also a significant decrease of the relative power of Delta brainwaves as compared to the baseline.

When comparing the baseline state to the brain state following ingestion of the mixture, a statistically significant 6.4% decrement of the Delta Relative Power (p<0.01%) was observed. This slowest of brainwave frequencies is most closely associated with sleep states. Therefore a reduction of delta band power is widely interpreted as a more awake or alert brain state.

When comparing the baseline state to the brain state following ingestion of the mixture, a statistically significant (p<0.01%) 16% increase of the gamma relative power was observed. Gamma waves are widely known to reflect higher cognitive activity. Gamma waves play an important role in processing information and problem solving-two key features of intelligence. Conversely, the overwhelming evidence in dementia research concludes that impaired cognitive states are tightly linked to reduced gamma power activity.

Moreover, the cortical areas that showed the greatest increase of gamma activity were the parietal lobe regions. Gamma wave activity in the parietal lobes specifically is associated with improved perception and cognitive coordination. Improved cognitive coordination is a fundamental activity that determines how well different regions of the brain are working together.

When comparing the baseline state to the brain state following exogenous ketone ingestion, a statistically significant increase of the alpha relative power (p<0.01%) was observed. There was also better modulation (sometimes described as ‘organization’) of the alpha rhythm seen by visual inspection of the raw EEG background activity. Alpha waves are associated with focus, flow state, and creativity; and alpha band frequencies seem to also play an important role in learning. Therefore, a substantial increase in alpha band power is widely interpreted as facilitating a more alert, but relaxed brain-state. In this study, we measured a sizable increase of alpha by 17%. Alpha frequency power and organization have been shown to be degraded or reduced across many disparate psychiatric and neurological diseases such as schizophrenia, bipolar disorder, and Alzheimer's dementia.

In one analysis of 2 large datasets on brain network stability, the researchers concluded that destabilization of brain networks reflects early signs of impaired metabolism seen in dementia states. In their study of exogenous ketones, they found that glucose utilization was improved by either a ketogenic diet or exogenous ketone ester ingestion (Mikicin, M., et al. (2015). Brain-training for physical performance: a study of EEG-neurofeedback and alpha relaxation training in athletes. Acta neurobiologiae experimentalis, 75(4), 434-445).

Investigated a group of female soccer players using QEEG and found a robust correlation between their athletic performance and certain QEEG parameters correlated to decision-making and anxiety. They concluded that this method of functional brain analysis is a reliable tool for predicting performance levels (Tharawadeepimuk, K., & Wongsawat, Y. (2017). Quantitative EEG evaluation for performance level analysis of professional female soccer players. Cognitive neurodynamics, 11(3), 233-244). In another study of dynamic peripheral visual performance among soccer players, the researchers postulated that up-regulation or augmenting alpha activity could be reasonably expected to improve the visual performance skill (Nan et al., (2014). Dynamic peripheral visual performance relates to alpha activity in soccer players. Frontiers in human neuroscience, 8, 913).

Alpha brain training has been widely used and studied as a technique for improving athletic performance. While the QEEG is the assessment tool, the intervention or therapeutic approach to improving alpha activity is called EEG-guided neurofeedback, or simply neurofeedback (NFB). Mikicin et al. studied a group of athletes, half of whom underwent NFB training sessions. The trained group exhibited greater reduction of reaction times in a test of visual attention versus the control group and showed improvement in several performance measures of Kraepelin's work-curve, used to evaluate speed, effectiveness and work accuracy. Together, these results supported the use of holistic, neurophysiological training in sports workout.

In summary, several metrics of optimal brain function improved following ingestion of the uniquely formulated mixture of the present disclosure. Bio-electrical activity measured by QEEG demonstrated a robust reduction of delta and increase in alpha and gamma band frequencies.

Those skilled in the field will understand based on the present disclosure that the various examples above may be scaled and altered to achieve desired results to serve specific purposes. For example, the recipes above may be scaled up for commercial purposes and adjustments may be made to accommodate production on large scale equipment such as adjustments to time, temperature, and amounts of materials without departing from the scope and spirit of the present disclosure.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

### REFERENCES

1. Mochel F, et al. J Neurol Neurosurg Psychiatry (2016); 87:550-553.
2. Fanny Mochel. “Triheptanoin for the treatment of brain energy deficit: A 14-year experience.” Journal of Neuroscience Research, (2017); 95 (11):2236-2243.
3. Roe C R, Sweetman L, Roe D S, David F, Brunengraber H. “Treatment of cardiomyopathy and rhabdomyolysis in long-chain fat oxidation disorders using an anaplerotic odd-chain triglyceride.” J Clin Invest (2002); 110:259-269.
4. Wehbe, Z, Tucci, S. “Therapeutic potential of triheptanoin in metabolic and neurodegenerative diseases.” J Inherit Metab Dis. (2020); 43: 385-391.
5. Marin-Valencia et al., Journal of Cerebral Blood Flow & Metabolism (2013); 33:175-182.
6. Ryoji Noyori, et al.: Asymmetric hydrogenation of .beta.-keto carboxylic esters. A practical, purely chemical access to .beta.-hydroxy esters in high enantiomeric purity. J.

Am. Chem. Soc., 1987, 109 (19), pp 5856-5858.
7. Park S J, Lee S Y, Lee Y (2004) Biosynthesis of (R)-3-hydroxyalkanoic acids by metabolically engineered Escherichia coli. Appl Biochem Biotechnol 113:373-379.
8. Westman, Eric C.; Tondt, Justin; Maguire, Emily; Yancy, William S. (15 Sep. 2018). “Implementing a low-carbohydrate, ketogenic diet to manage type 2 diabetes mellitus”. Expert Review of Endocrinology & Metabolism. 13 (5): 263-272. doi:10.1080/17446651.2018.1523713. PMID 30289048. S2CID 52920398.
9. Ketogenic diet improves metabolic syndrome in multiple ways, diabetes.co.uk, December 2017.
10. Gershuni, Victoria M.; Yan, Stephanie L.; Medici, Valentina (20 Aug. 2018). “Nutritional Ketosis for Weight Management and Reversal of Metabolic Syndrome”. Current Nutrition Reports. 7 (3): 97-106. doi:10.1007/s13668-018-0235-0. PMC 6472268. PMID 30128963.
11. Mattson M P, Moehl K, Ghena N, Schmaedick M, Cheng A (2018). “Intermittent metabolic switching, neuroplasticity and brain health”. Nature Reviews. Neuroscience. 19 (2): 63-80. doi:10.1038/nrn.2017.156. PMC 5913738. PMID 29321682.
12. Ward C (2015). “Ketone Body Metabolism”. Diapedia. doi:10.14496/dia.51040851169.29.
13. Mujica-Parodi L R, Amgalan A, Sultan S F, Antal B, Sun X, Skiena S, Lithen A, Adra N, Ratai E M, Weistuch C, Govindarajan S T, Strey H H, Dill K A, Stufflebeam S M, Veech R L, Clarke K. Diet modulates brain network stability, a biomarker for brain aging, in young adults. Proc Natl Acad Sci USA. 2020 Mar. 17; 117(11):6170-6177. doi: 10.1073/pnas.1913042117. Epub 2020 Mar. 3. PMID: 32127481; PMCID: PMC7084077.
14. Fortier M, Castellano C A, Croteau E, Langlois F, Bocti C, St-Pierre V, Vandenberghe C, Bernier M, Roy M, Descoteaux M, Whittingstall K, Lepage M, Turcotte ÉE, Fulop T, Cunnane S C. A ketogenic drink improves brain energy and some measures of cognition in mild cognitive impairment. Alzheimers Dement. 2019 May; 15(5):625-634. doi: 10.1016/j.jalz.2018.12.017. Epub 2019 Apr. 23. PMID: 31027873.
15. Fortier M, Castellano C A, Croteau E, Langlois F, Bocti C, St-Pierre V, Vandenberghe C, Bernier M, Roy M, Descoteaux M, Whittingstall K, Lepage M, Turcotte E E, Fulop T, Cunnane S C. A ketogenic drink improves brain energy and some measures of cognition in mild cognitive impairment. Alzheimers Dement. 2019 May; 15(5):625-634. doi: 10.1016/j.jalz.2018.12.017. Epub 2019 Apr. 23. PMID. 31027873.
16. Machado C, Cuspineda E, Valdes P, et al. Assessing acute middle cerebral artery ischemic stroke by quantitative electric tomography. Clin EEG Neurosci 2004, 35:116-124.
17. Machado C, Estevez M, Perez-Nellar J, et al. Autonomic, EEG, and behavioral arousal signs in a PVS case after Zolpidem intake. Can J Neurol Sci 2011; 38:341-344.
18. Machado C, Estevez M, Carrick F, Mellilo R, Leisman G. qEEG may increase the reliability of diagnostic and prognostic procedures in cerebral arterial gas embolism. Clin Neurophysiol 2012; 123:225-226.
19. Machado C, Estevez M, Leisman G, et al. QEEG spectral and coherence assessment of autistic children in three different experimental conditions. J Autism Dev Disord 2015; 45:406-424.
20. Machado C, Korein J, Aubert E, et al. Recognizing a mother's voice in the persistent vegetative state. Clin EEG Neurosci 2007:38:124-126.
21. Poff, A. M., Moss, S., Soliven, M., & D'Agostino, D. P. (2021). Ketone Supplementation: Meeting the Needs of the Brain in an Energy Crisis. Frontiers in nutrition, 8, 783659.
22. Svart, M., Gormsen, L. C., Hansen, J., Zeidler, D., Gejl, M, Vang, K., Aanerud J., & Moeller, N. (2018). Regional cerebral effects of ketone body infusion with 3-hydroxybutyrate in humans: Reduced glucose uptake, unchanged oxygen consumption and increased blood flow by positron emission tomography. A randomized, controlled trial. PloS one, 13(2), e0190556.
23. Fortier, M., Castellano, C.-A., Croteau, E., Langlois, F., Bocti, C., St-Pierre, V, Vandenberghe, C., Bernier, M, Roy, M, Descoteaux, M, Whittingstall, K, Lepage, M., Turcotte, É. E., Fulop, T. and Cunnane, S. C. (2019), A ketogenic drink improves brain energy and some measures of cognition in mild cognitive impairment. Alzheimer's & Dementia, 15: 625-634.
24. Traikapi A, Konstantinou N. Gamma Oscillations in Alzheimer's Disease and Their Potential Therapeutic Role. Front Syst Neurosci. 2021; 15:782399. Published 2021 Dec. 13.
25. Ba,sar, Erol, G″untekin, Bahar, A short review of alpha activity in cognitive processes and in cognitive impairment, International Journal of Psychophysiology (2012), doi: 10.1016/j.ijpsycho.2012.07.001
26. Mikicin, M., Orzechowski, G., Jurewicz, K., Paluch, K., Kowalczyk, M., & Wrobel, A. (2015). Brain-training for physical performance: a study of EEG-neurofeedback and alpha relaxation training in athletes. Acta neurobiologiae experimentalis, 75(4), 434-445.
27. Tharawadeepimuk, K., & Wongsawat, Y. (2017). Quantitative EEG evaluation for performance level analysis of professional female soccer players. Cognitive neurodynamics, 11(3), 233-244.
28. Nan, W., Migotina, D., Wan, F., Lou, C. I., Rodrigues, J., Semedo, J., Vai, M. I., Pereira, J. G., Melicio, F., & Da Rosa, A. C. (2014). Dynamic peripheral visual performance relates to alpha activity in soccer players. Frontiers in human neuroscience, 8, 913.
29. Dr. David Sortino, Neurofeedback: How to get into the zone, davidsortino.blogs.pressdemocrat.com/10244/neurofeedback-how-to-get-into-the-zone.

1. A process for producing optically active (R)-3-hydroxybutyrate and (R)-3-hydroxyvalerate mixtures from biomass containing poly-3-hydroxybutyrate-co-3-hydroxyvalerate (PHB-co-HV), comprising:
a) adjusting the 3-hydroxyvalerate content in the PHB-co-HV polymer between 0 and 40%, by controlled addition of sugars and one or more precursor compounds in a fermentation phase of the PHB-co-HV;
b) extracting PHB-co-HV from biomass by the addition of one or more proteolytic enzymes;
c) adding an alcohol and an acidifying agent to the extracted PHB-co-HV to form a suspension;
d) heating the suspension to a temperature of 70 to 150° C. to form a solution;
e) neutralizing the formed solution with an alkalizing agent in a reaction;
f) distilling the mixture of (R)-3-hydroxybutyrate/(R)-3-hydroxyvalerate esters under vacuum to remove excess alcohol and form a distilled mixture;
g) adding an alkalizing agent to the distilled mixture; and
h) removing volatile compounds by vacuum distillation, and collecting the final mixture (R)-3-hydroxybutyrate and (R)-3-hydroxyvalerate.

2. The process of claim 1, where the precursors used in the fermentative phase of the synthesis of PHB-co-HV(a) are chosen from propionic acid, valeric acid, isopropanol, n-propanol and n-pentanol.

3. The process of claim 1, where the proteolytic enzymes used in the extraction step of PHB-co-HV from the biomass (b) are bacterial or fungal proteases capable of acting in an acidic environment.

4. The process of claim 1, where the alcohol that is added to the PHB-co-HV for the step hydrolyzing the polymer chain and trans-esterification (c) is methanol, ethanol, n-propanol or isopropanol.

5. The process of claim 4, where the amount of alcohol added is equivalent to 2 to 10 times a stoichiometrically necessary amount for the formation of the ester.

6. The process of claim 1, where the acidifying agent used in the trans-esterification step (c) is HCl, H₂SO₄ or organic tin salts, in particular tin oxalate.

7. The process of claim 6, wherein the acidifying agent is added in an amount equivalent to 1% and up to 10% by weight of added PHB-co-HV.

8. The process of claim 1, where the alkalizing agent, used in the neutralization step (e) after trans-esterification is NaOH, KOH, Ca(OH)₂, Na₂CO₃, NaHCO₃, K₂CO₃ or KHCO₃.

9. The process of claim 1, wherein the excess alcohol from the trans-esterification reaction, (f), is removed by distillation, rectified and dehydrated in order to return to the process.

10. The process of claim 1, wherein such mixture is further purified by fractional vacuum distillation, at a temperature between about 80° C. and 120° C., under an absolute pressure between about 5 and about 50 mmHg.

11. The process of claim 1, wherein the mixture of (R)-3-hydroxybutyrate/esters/(R)-3-hydroxyvalerate is subjected to an alkalization process with the addition of sodium, potassium, calcium or magnesium hydroxides, or a mixture thereof, leading to the breakdown of the esters and formation of their respective salts.

12. The process of claim 1, wherein the mixture of (R)-3-hydroxybutyrate and (R)-3-hydroxyvalerate salts is subjected to a concentration and purification step by evaporation at a temperature between 50° C. and 70° C., under an absolute pressure of about 5 to 50 mmHg.

13. The process of claim 12, wherein the mixture of (R)-3-hydroxybutyrate and (R)-3-hydroxyvalerate salts is passed through a bed of cationic resins so that the counter-ions sodium, potassium, magnesium and/or calcium are replaced by H+, forming the respective acids (R)-3-hydroxybutyric and (R)-3-hydroxyvaleric.

14. A method of treating a subject having a metabolic disorder, comprising administering optically active mixtures of (R)-3-hydroxybutyrate and (R)-3-hydroxyvalerate, their respective salts or esters derived therefrom, obtained by the process of claim 1, at a therapeutically effective amount to treat at least one symptom of the metabolic disorder.

15. The method of treatment of claim 14, wherein the metabolic disorder comprises deficiency of glucose transporter 1 (GLUT1-DS).

16. The method of claim 14, wherein the subject has a central nervous system disorder.

17. The method of claim 16, wherein the central nervous system disorder is epilepsy, Huntington's disease, Parkinson's disease, Alzheimer's disease, senile dementia, Pick's disease, or Cretzfeldt-Jacobs' disease.

18. The method of claim 14, where these metabolic disorder comprises deficiency of pyruvate carboxylase (PC).

19. The method of claim 14, comprising administering a combination of (R)-3-hydroxybutyrate and (R)-3-hydroxyvalerate or (R)-3-hydroxybutyrate, (R)-3-hydroxyvalerate, and (D)-1,3 butanediol.

20. The method of claim 14, comprising administering a combination of (R)-3-hydroxybutyrate and (R)-3-hydroxyvalerate or (R)-3-hydroxybutyrate, (R)-3-hydroxyvalerate, and (D)-1,3 butanediol in an amount of 0.10 to 1 g/kg, 0.12 to 0.8 g/kg, 0.13 to 0.7 g/kg, 0.14 to 0.6 g/kg, or 0.15 to 0.5 g/kg per unit dosage.

21. The method of claim 14, comprising administering a combination of (R)-3-hydroxybutyrate and (R)-3-hydroxyvalerate or (R)-3-hydroxybutyrate, (R)-3-hydroxyvalerate, and (D)-1,3 butanediol at a dosage of 0.30 to 3 g/kg, 0.4 to 2.5 g/kg, 0.5 to 2 g/kg, 0.75 to 1.5 g/kg, 0.8 to 1 g/kg per day.

22. The method of claim 14, wherein the proportion of (R)-3-hydroxybutyrate to (R)-3-hydroxyvalerate is 1.0 to 0 and 0.7 to 0.3, 0.99 to 0.01, 0.98 to 0.02, 0.97 to 0.03, 0.96 to 0.04, 0.95 to 0.05, 0.9 to 0.1, 0.85 to 0.15, 0.8 to 0.2, or 0.75 to 0.25.

23. The method of claim 14, wherein the subject achieves a circulating ketone level of about 1.3, 1.4, 1.5, 1.6, 1.7, 1.8, 1.9, 2.0, 2.1, 2.2, 2.3, 2.4, 2.5 mM or higher.

24. The method of claim 14, wherein the subject achieves an increase in circulating ketone level from baseline (prior to administration) of at least 0.5 mM.

25. The method of claim 14, wherein the unit dosage includes 5-50 grams of the combination.

26. The method of claim 14, comprising administering at least 10 grams of the combination daily or multiple times per day.

27. The method of claim 14, comprising administering at least 5 grams of the combination every 2, 3, 4, or 5 hours.

28. The method of claim 14, wherein alpha brain waves are increased by at least 10% after the administering step as measured by Quantitative Electroencephalogram (QEEG).

29. The method of claim 14, wherein alpha brain waves are increased by at least 15% after the administering step as measured by Quantitative Electroencephalogram (QEEG).

30. The method of claim 14, wherein gamma brain waves are increased by at least 10% after the administering step as measured by Quantitative Electroencephalogram (QEEG).

31. The method of claim 14, wherein gamma brain waves are increased by at least 15% after the administering step as measured by Quantitative Electroencephalogram (QEEG).

32. The method of claim 14, wherein delta brain waves are decreased by at least 5% after the administering step as measured by Quantitative Electroencephalogram (QEEG).
